(19) United States
(12) Patent Application Publication
(10) Pub. No.: US 2023/0296558 A1
(43) Pub. Date: Sep. 21, 2023

(54) HYDROGEL-BASED PACKAGING OF 2D MATERIALS-BASED BIOSENSOR DEVICES FOR ANALYTE DETECTION AND DIAGNOSTICS

(71) Applicant: Grolltex, Inc., San Diego, CA (US)

(72) Inventor: Aliaksandr Zaretski, San Diego, CA (US)

(21) Appl. No.: 18/015,271

(22) PCT Filed: Jul. 14, 2021
(86) PCT No.: PCT/US2021/041570

§ 371 Date: Jan. 9, 2023

Related U.S. Application Data
(60) Provisional application No. 63/051,638, filed on Jul. 14, 2020.

(51) Int. Cl. G01N 27/447

(52) U.S. Cl. CPC G01N 27/44791; G01N 27/44747

(57) ABSTRACT

A biosensor device for detecting an analyte in a sample, and methods of fabrication and use thereof. The biosensor device includes a vertical stack including a patterned biosensor layer; a hydrogel layer disposed above and in contact with the patterned biosensor layer; a permeable metallic electrode disposed above the hydrogel layer; and a sample collection layer disposed proximate and in contact with the permeable metallic electrode.

### RELATED APPLICATION

This application claims priority to and the benefit of U.S. Provisional Pat. Application No. 63/051,638, filed July 14. 2020, the entire contents of which are incorporated by reference herein.

### BACKGROUND

Detecting biological molecules relevant to diagnosing infectious and other diseases in biological substances (e.g., saliva) is currently difficult. The concentrations of the analytes (e.g., viral RNA) are typically exceedingly small. Additionally, saliva is host to a large number of pathogens and food debris that can contaminate sensors and cause fouling.

Existing biosensors are generally not sufficiently sensitive to detect trace amounts of disease-related biomolecules present in saliva.

### SUMMARY

Hydrogel-based packaging of 2D materials-based biosensor devices for analysis and diagnostics are described herein. By incorporating electrophoresis into a biosensor device in accordance with embodiments of the invention, charged analytes can be propelled to the sensor surface with an electric field, thus significantly increasing their concentration at the surface. The process of electrophoresis simultaneously removes oppositely charged molecules and particles from the sensor surface as well as filters out particles larger than the hydrogel pore size and thus protects the sensors from fouling. Additional advantages are provided by incorporating a sample collection layer into the same vertical stack in which electrophoresis is conducted. Moreover, the described biosensor device enables molecular diagnostics with a disposable device, with sample-to-answer achieved with a single device. In addition, sample preparation (e.g., collection, filtration, deionization, lysis, concentration, heating, and decontamination) may all take place in a single device.

In an aspect, embodiments of the invention relate to a biosensor device for detecting an analyte in a sample. The biosensor device includes a vertical stack including a patterned biosensor layer; a hydrogel layer disposed above and in contact with the patterned biosensor layer; a permeable metallic electrode disposed above the hydrogel layer; and a sample collection layer disposed proximate and in contact with the permeable metallic electrode.

One or more of the following features may be included. The patterned biosensor layer may include a nanocarbon material, graphene with noble metal nanoislands formed thereon, a transition-metal dichalcongenide (TMD), a two-dimensional (2D) material, and/or a one-dimensional material.

The hydrogel layer may include agarose and/or polyacrylamide.

The hydrogel layer may be adapted to electrophoretically filter and separate a plurality of sample moieties by size.

The hydrogel layer may be adapted to electrophoretically concentrate a plurality of oligonucleotides from the sample at a surface of the patterned biosensor layer.

The permeable metallic electrode may be adapted to control a temperature of the biosensor device. The temperature may result in lysis of the sample, and/or enhance hybridization of the analyte to a biomolecular ligand immobilized in contact with the patterned biosensor layer.

The permeable metallic electrode may be configured to act as a cathode and/or an anode during electrophoresis through the hydrogel layer.

The patterned biosensor layer may be disposed above and in contact with a substrate. The substrate includes glass, a polymeric film, a single crystal material, surface-enhanced Raman Spectroscopy (SERS) substrate, a localized surface plasma resonance (LSPR) substrate, a surface plasma resonance (SPR) substrate, fluorescence in situ hybridization (FISH) labeled substrate, and/or an electrochemical sensing substrate.

The biosensor device may further include a biomolecular ligand immobilized over and in contact with the patterned biosensor layer. The biomolecular ligand may include a plurality of oligonucleotide probes, antibodies, antigens, and/or enzymes.

The sample collection layer may include an absorbent filter medium, and/or a matrix.

The sample collection layer may be disposed above the permeable metallic electrode.

The biosensor device may further include an absorption layer disposed between the hydrogel layer and the permeable metallic electrode.

A dongle may be in electrical communication with the biosensor device. The dongle may include a housing; a power supply disposed within the housing; at least one electrode probe in electrical communication with the power supply and configured to make electrical contact to the patterned biosensor layer.

The dongle may further include a spring configured to, in a first position, position the electrode probe(s) to contact the patterned biosensor layer, and, in a second position, to retract the electrode probe(s). The dongle may include an interface for electrical communication with a computing device.

In another aspect, embodiments of the invention relate to a method for fabricating a biosensor device. The method includes fabricating a vertical stack by providing a patterned biosensor layer; forming a hydrogel layer over and in contact with the patterned biosensor layer; forming a permeable metallic electrode over the hydrogel layer; and forming a sample collection layer proximate and in contact with the permeable metallic electrode.

One or more of the following features may be included. A biomolecular ligand may be immobilized over and in contact with the patterned biosensor layer. At least two different types of biomolecular ligands may be immobilized over and in contact with the patterned biosensor layer. Immobilizing the at least two different types of biomolecular ligands may include a) applying at least two different types of capping agents to the patterned biosensor layer, b) selectively removing one of the capping agents. c) immobilizing a biomolecular ligand to the patterned biosensor layer, d) repeating steps b) and c) at least once with a different type of biomolecular ligand.

In yet another aspect, embodiments of the invention relate to a method for detecting an analyte in a sample. The method includes the steps of depositing the sample onto a top surface of a biosensor device; inserting the biosensor device into a measuring device; activating the measuring device to induce the biosensor device to generate a signal indicating at least one of a presence or a quantity of the analyte; and transmitting data describing the signal from the measuring device to a computing device.

One or more of the following features may be included. The biosensor device may include a patterned biosensor layer, a hydrogel layer disposed above and in contact with the patterned biosensor layer, a permeable metallic electrode disposed above the hydrogel layer, and a sample collection layer disposed proximate and in contact with the permeable metallic electrode.

Activating the measuring device may include applying an electrical signal to the biosensor device and/or illuminating the biosensor device.

The measuring device may include a dongle. The computing device may include a smartphone.

### BRIEF DESCRIPTION OF DRAWINGS

FIG. 1 is an exploded view of a layer structure for a biosensor device, in accordance with an embodiment of the invention;

FIG. 2 is an exploded view of a layer structure for a biosensor device, in accordance with an embodiment of the invention;

FIG. 3 is a perspective view of a device structure for a biosensor device, in accordance with an embodiment of the invention;

FIG. 4 is a perspective view of a biosensor device, in accordance with an embodiment of the invention;

FIG. 5 is a perspective view of a biosensor device inserted into a dongle and an exploded view of the biosensor device with electrode probes of the dongle extending therethrough, in accordance with an embodiment of the invention:

FIG. 6 is a perspective view of a biosensor device inserted into a dongle and into a smartphone, in accordance with embodiments of the invention;

FIG. 7 is a flowchart and schematic illustration of a method for fabricating a biosensor device, in accordance with an embodiment of the invention;

FIGS. 8A and 8B are exploded views of a biosensor device in use, in accordance with embodiments of the invention;

FIG. 9 is a perspective view of a screening unit employing a biosensor device, in accordance with an embodiment of the invention;

FIG. 10 is a schematic illustration of complementary RNA hybridized to immobilized DNA in a biosensor device in accordance with an embodiment of the invention;

FIG. 11 is a graph illustrating the resulting electrical signal of the RNA hybridized to DNA in FIG. 10;

FIG. 12 is a graph illustrating COVID oligonucleotide detection, in accordance with an embodiment of the invention: and

FIG. 13 is a graph illustrating Dirac point shift, in accordance with an embodiment of the invention.

### DETAILED DESCRIPTION OF THE INVENTION

Detecting biological molecules relevant to diagnosing infectious and other diseases in saliva is currently difficult. The concentrations of the analytes (e.g., viral RNA) are typically exceedingly small. Additionally, saliva is host to a large number of pathogens and food debris that can contaminate sensors and cause fouling. Embodiments of the invention disclosed herein address this challenge.

By adding a thin layer of a hydrogel (e.g.. agarose) capped with a liquid permeable and electrically conductive material (e.g., metal mesh/filter paper composite) to a sensor surface, it is possible to collect the saliva, filter it, and electrophoretically separate the analytes and deliver them to the sensor surface for analysis.

Embodiments of the described device provide an advanced biosensor storage and stabilization solution as well as the ability to filter and concentrate the analytes at the sensor surface. The approach significantly enhances both the sensitivity and selectivity of 2D materials-based biosensors, which are easily fouled by proteins, pathogens, and food debris, thereby leading to loss of sensitivity.

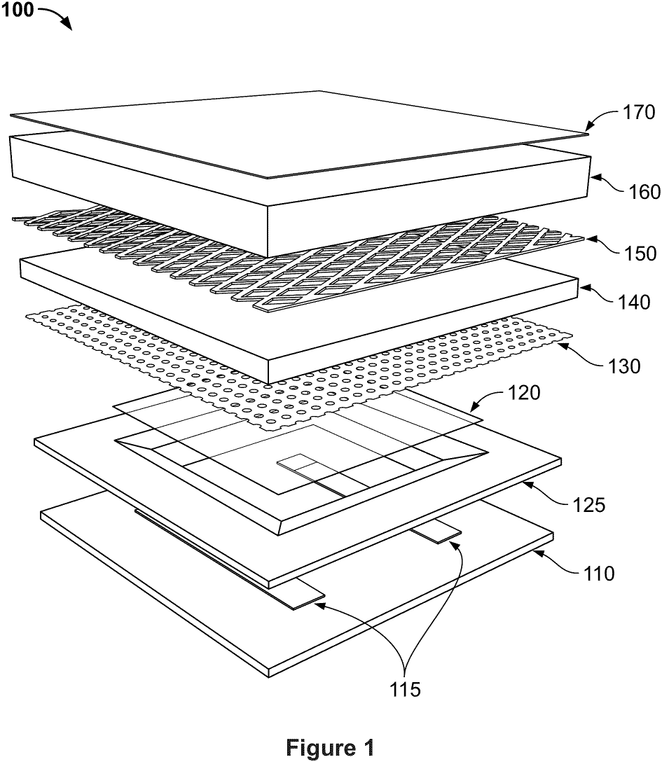

Referring to FIG. 1, in accordance with an embodiment of the invention, a biosensor device, also referred to herein as a chip, includes a vertical layer structure 100. The layer structure 100 may include a substrate 110 that is, in certain embodiments, transparent, lenticular, and chemically resistant. For example, in some embodiments, the substrate 110 is a transparent chemically resistant plastic subjected to a proprietary treatment that allows graphene transfer as well as reduces graphene doping from overlying layers (i.e., the Dirac point remains around 0 V, with no gating required for sensing and allowing a two-electrode approach). In some embodiments, the substrate is transparent and lenticular, which enables optical investigation through the bottom of a chip.

In some embodiments, the substrate 110 includes glass, a polymeric film, or a single crystal material. A suitable glass is, e.g., borosilicate, soda lime glass, or flexible glass, such as Corning WILLOW glass. A suitable polymeric film is, e.g., polyethylene terephthalate (PET), polyimide, polyethylene naphthalate (PEN), poly(methylmethacrylate) (PMMA), polycarbonate (PC), an elastomer polymer, a thermoplastic polymer, and/or a chemical vapor deposition (CVD)-deposited polymer (ex.: Parylene-C or D or N). A suitable single crystal material is, e.g., silicon.

In some embodiments, the substrate 110 is a flexible substrate. Flexible substrates include, for example, polyethylene terephthalate (PET), polyimide, polyethylene naphthalate (PEN), polycarbonate (PC), an elastomer polymer, and combinations thereof. The elastomer polymer may be transparent. Examples of a suitable elastomer polymer include polydimethylsiloxane (PDMS), or silicone rubber. The flexible substrate may be, for example, a transparent flexible substrate.

In some embodiments, the substrate is a rigid substrate, such as, for example, a glass substrate, a Si substrate, an oxide substrate such as a silicon dioxide (SiO₂) substrate or indium tin oxide (ITO) substrate, tin oxide (SnO) substrate, a titanium oxide (TiO) substrate, or an aluminum oxide (Al₂O₃) substrate, and/or a metal substrate. Suitable metal substrates may include at least one metal or alloy selected such as copper (Cu), nickel (Ni), cobalt (Co), iron (Fe), platinum (Pt), gold (Au), ruthenium (Ru), and/or aluminum (Al). An oxide substrate may include an oxide of metal having an insulating property, a conductive property, or a semiconductor property.

In various embodiments, the substrate 110 may be a surface-enhanced Raman spectroscopy (SERS) or localized surface plasmon resonance (LSPR) substrate including, e.g., gold nanoparticles: a surface plasmon resonance (SPR) substrate including, e.g., gold thin films, a fluorescence in situ hybridization (FISH) labeled substrate, and/or an electrochemical sensing substrate including. e.g., gold and graphene ink electrodes.

Referring still to FIG. 1, a patterned biosensor layer 115 may be disposed on the substrate 110. In some embodiments, the patterned biosensor layer may include graphene with nanoislands of noble metals formed thereon. The graphene layer may have a thickness of, e.g., 0.3 nm - 10 nm. The nanoislands may have a thickness of, e.g., 0.2 nm -15 nm. Suitable noble metals include, e.g., gold, palladium, silver, platinum, osmium, iridium, rhodium, and ruthenium. In addition or alternatively, the patterned biosensor layer may include nanotubes (thickness of. e.g., 1 nm - 100 nm), graphene-oxide flakes (thickness of, e.g., 0.3 nm - 100 nm), and/or reduced graphene-oxide (thickness of, e.g.. 0.3 nm - 100 nm). Advantages of the patterned biosensor layer including nanoislands of noble metals on graphene may include: 1) increased biosensor surface area (improved sensitivity via higher probe density); 2) facile and robust ligand binding chemistry (improved manufacturing process/storage stability); 3) sensor surface topography with an extended Debye-layer (improves sensitivity): and 4) optionally, enablement of optical investigation of the biosensor via plasmonic schemas, e.g.. via SPR, LSPR, and/or Raman spectroscopy.

In other embodiments, the patterned biosensor layer 115 includes transition-metal dichalcogenides (TMDs) (e.g., MoS₂, WSe₂, and/or CuS) (thickness of. e.g., 0.3 nm - 10 nm), two-dimensional (2D) materials (for example, phosphorene and/or silicene) (thickness of, e.g., 0.3 nm -10 nm), and/or one-dimensional (1D) materials such as Si nanowires (thickness of, e.g., 1 nm - 100 nm).

In some embodiments, biomolecular ligand may be immobilized on the biosensor layer 115 surface. The biomolecular ligand may include, e.g.. oligonucleotide probes, antibodies, antigens, and/or enzymes. In some embodiments, a composition of the biomolecular ligand is selected such that the ligand’s composition is complementary to the analyte that is to be detected.

Referring still to FIG. 1, a hydrogel layer 120 is disposed above and in contact with the patterned biosensor layer 115 for mechanical and electrophoretic separation of analytes. The hydrogel layer 120 may include, e.g., agarose and/or polyacrylamide. In some embodiments, the hydrogel layer 120 may be pre-charged with optionally embedded nanoparticles conjugated with target-specific probes (with sequences different from probes on the biosensor layer surface). In some embodiments, the composition of such probes includes gold nanoparticles (maximum dimension of, e.g., 2 nm - 100 nm), quantum dots (CdTe, maximum dimension of, e.g., 2 nm - 9 nm), and/or fluorescent nanobeads (polyacrilnitrile, maximum dimension of, e.g.. 5 nm - 100 nm) labeled with fluorescence resonance energy transfer (FRET) dye.

These probes may decorate a target RNA analyte and further enhance a signal. For example, the hydrogel 120 may include a low concentration gel matrix (agarose or polyacrylamide) suitable for mechanical and electrophoretic filtration and separation of saliva, as well as for hosting the embedded nanoparticles (e.g.. gold, quantum dots) conjugated with target-specific probes (with sequences different from the probes on the sensor surface).

In particular, the hydrogel layer may be adapted to electrophoretically filter and/or separate a plurality of sample moieties by size. i.e., nanopores of the hydrogel enable such filtration and/or separation. In some embodiments, the hydrogel layer is adapted to electrophoretically concentrate a plurality of oligonucleotides from a sample at the surface of the patterned biosensor layer during operation of the biosensor device, as discussed in more detail below. Advantages of the hydrogel layer 120 may include: 1) protective encapsulation of the biosensor and stabilization of the oligonucleotide probes on its surface; 2) electrophoretic filtering and separation of the sample moieties by charge; and 3) electrophoretic concentration of oligonucleotides from a sample at the surface of the biosensor layer.

Referring still to FIG. 1,a spacer 125 defining a gel window may surround the hydrogel layer 120. The spacer 125 may facilitate the placement of the hydrogel layer 120 over the biosensor layer as described below, as well as maintain the hydrogel layer 120 in place. In some embodiments, the spacer 125 may include a plastic layer, such as TEFLON, PEEK, PEN, PET, and/or PMMA [thickness of, e.g.. 10 micrometers (µm) - 100 µm]. The spacer 125 may define a window to hold the hydrogel layer 120. with the hydrogel layer 120 being operatively connected to the biosensor layer 115.

Referring still to FIG. 1, a membrane filter 130 may be disposed above the hydrogel layer 120 and the spacer 125. In some embodiments, the membrane filter 130 includes a track-etched membrane to separate the hydrogel layer 120 from an absorption layer 140 in storage and/or to ensure proper gel hydration. In some embodiments, a membrane filter may include polycarbonate or polyester (thickness of, e.g., 3 µm - 100 µm) with pore diameter of, e.g., 0.2 µm - 10 µm and open area between 10% and 90%.

In some embodiments, the membrane filter 130 includes a slightly hydrophilic membrane, with the surface being wettable but not hygroscopic so that the hydrogel water may not be pulled through it to the absorption layer via capillary action in storage, and such that the surface does not resist the flow of saliva and the analytes it contains upon actuating the device. In some embodiments, the slightly hydrophilic membrane includes a water droplet angle (or wetting angle) between 75° and 105°. The membrane filter 130 may have a thickness ranging from 3 µm to 100 µm, e.g.. it may have a thickness of about 25 µm. The membrane filter 130 is preferably thick enough so that the hydrogel water is not pulled through it to the absorption layer via capillary action in storage, while being thin enough to not resist the flow of saliva upon actuating the device.

In some embodiments, the membrane filter 130 includes pores having an inner diameter ranging from 0.1 µm to 10 µm, e.g., 5 µm. The pores are preferably large enough such that the hydrogel water is not pulled through the membrane filter to the absorption layer via capillary action in storage, while being small enough to not resist the flow of saliva upon actuating the device.

The membrane filter 130 may include from 1% to 99% open area, e.g., 25% to 75% or about 50% open area. The membrane preferably has sufficient open area such that it does not resist the flow of saliva upon actuating the device., and not so much open area that hydrogel water is not pulled through the membrane filter to the absorption layer via capillary action in storage.

Referring still to FIG. 1, the porous absorption layer 140 is disposed above the membrane filter 130 for providing lysis-assisting compounds. The absorption layer 140 may include a cellulosic matrix. e.g., cellulose, methyl cellulose, ethyl-cellulose, etc., and combination thereof or nylon, etc. The absorption layer may include a low affinity cellulose-based layer that is pre-charged with guanidine salts and non-ionic surfactants. For example, the absorption layer 140 may be pre-charged with guanidine thiocyanate (GITC) and/or surfactant for lysing. Suitable materials for the absorption layer 140 may be hygroscopic, non-adsorbent to biomolecules, and porous.

The absorption layer 140 has a thickness of 100 µm to 2.5 mm, e.g.. 200 µm - 1 mm, such as 495 µm. The absorption layer is preferably thick enough (e.g., 500 micrometers) such that it can accept a desirable volume of sample (e.g.. 10 µl - 1 ml), and thin enough such that it reaches the saturation point with the available collected sample.

Referring still to FIG. 1, a permeable metallic electrode 150 is disposed above the absorption layer 140. In some embodiments, the permeable metallic electrode 150 may be a metal mesh electrode for electrophoresis uses. In some embodiments, the permeable metallic electrode is configured to act as a cathode and/or an anode during forward or reverse electrophoresis through the hydrogel layer.

In some embodiments, the permeable metallic electrode 150 is adapted to act as a heater to control a temperature of the biosensor device. Controlling the temperature of the biosensor device facilitates: 1) lysis of a sample; 2) enhancing the hybridization of a probe or target (for example, hybridization of analyte to a biomolecular ligand immobilized in contact with the patterned biosensor layer); 3) enhancing the specificity via keeping the temperature close to the specific melting point of a probe or target; and 4) autoclaving the biosensor device for safe disposal.

In some embodiments, the permeable metallic electrode 150 includes a perforated and expanded metal mesh electrode with 1% to 99% open area, e.g.. 50% - 95%, such as 75% open area. The permeable metallic electrode preferably has sufficient open area to allow the flow of saliva upon actuating the device., and without having so much open area that may provide non-uniform heating or electric field distribution.

In some embodiments, the permeable metallic electrode 150 has a thickness of 1 µm to 100 µm, e.g.. 5 µm to 45 µm, such as 25 µm. The permeable metallic electrode is sufficiently thick to provide adequate heating power, while being sufficiently thin such that it remains flexible and does not possess excessive thermal inertia. In some embodiments, the composition of the permeable metallic electrode includes aluminum, copper, gold, stainless steel, titanium, and/or alloys thereof.

Referring still to FIG. 1, a sample collection layer 160 is disposed above the permeable metallic electrode 150, and may be suitable for collecting saliva, absorbing saliva, and/or exchanging ions for further processing in the layers as described above. In some embodiments, the sample collection layer 160 includes, e.g., a polyurethane (PU) foam pad that may include low binding PU and may be an open-cell foam. In some embodiments, the sample collection layer 160 includes an absorbent filter medium. In some embodiments, the sample collection layer 160 includes a sample deionization matrix (for example, deionization resin and/or hydrogel). The sample deionization matrix allows instant deionization of the saliva samples to below about 1 mM ionic strengths, which provides advantages including: 1) enhancing sensitivity (for example, via extending the Debye layer): 2) allowing application of higher voltages in electrophoresis (for example, via faster transport kinetics) without inducing significant Faradaic currents and electrode degradation; and 3) enhancing cell lysis. In some embodiments, the sample deionization matrix includes a proprietary low binding affinity ion exchange matrix integrated into the PU foam pad, which may desalt saliva and achieve about 1 mM ionic strength within 10 seconds. In some embodiments, a low binding affinity ion exchange matrix includes pulverized (e.g., diameter of 100 nm to 100 µm) mixed bed (e.g.. cation and anion) ion exchange resin embedded in agarose (or acrylamide) matrix (e.g., 0.4%-4% agarose).

In some embodiments, the sample collection layer 160 has a thickness of 0.2 mm to 10 mm, e.g., 2 mm. The sample collection layer is sufficiently thick to accept a desirable volume of sample (e.g., a volume of 10 µl - 1 ml), and thin enough such that it can be placed into a mouth (in combination with the other layers of the device) and depressed with the tongue, not precluding easy sample collection.

Referring still to FIG. 1, a release liner 170 may be disposed above the sample collection layer 160. The release layer may be PET film with a thickness of 10 µm - 50 µm to protect the chip from fouling, drying out, and/or being contaminated in storage.

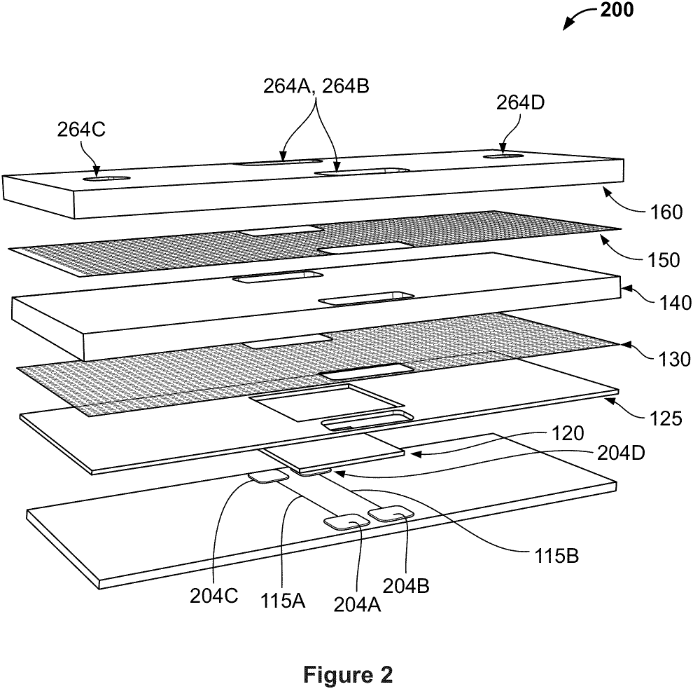

Referring to FIG. 2, a biosensor device 200, in accordance with an embodiment of the invention, includes a vertical layer structure 100. Advantages of a vertical structure, with the sample collection layer disposed directly over a hydrogel layer and biosensor layer, which are configured to facilitate analysis of a sample applied to the sample collection layer, include high sample flux/transport rate through the functional layers of the device resulting in rapid sample processing.

The patterned biosensor layer 115 on the substrate 110 may be patterned to define two channels 115A, 115B. In use, a first channel 115A may function as a biosensor channel and a second channel 115B may function as a control channel. As discussed above, the channels 115A, 115B may include graphene and/or nanoscopic gold particles (for example, nanoislands) to which oligonucleotide probes are conjugated.

In some embodiments, each channel 115A, 115B may have dimensions (e.g., a width and a length) of about 50 micrometers x 20 millimeters with further minimization possible. Accordingly, the channels may range from 5 micrometers x 0.1 millimeters to 100 micrometers x 40 millimeters. In some embodiments, each channel may include a rectangular surface with a longer dimension thereof ranging from 1 millimeter to 40 millimeters, e.g., 20 millimeters, and a shorter dimension ranging from 5 micrometers to 100 micrometers, e.g., 50 micrometers. The channels are preferably sufficiently large to enable facile fabrication and adequate functionalization as well as allow adequate current flow, and sufficiently small to adequately concentrate the analyte of interest as well as to allow placing multiples of such channels onto a single device.

In some embodiments, each channel 115A, 115B may have a thickness of about 10 micrometers with further minimization possible. Accordingly, the channels may range from 1 micrometer to 50 micrometers.

A proximal end of each channel 115A, 115B may be connected to a first metallic contact pad 204A, 204B, and a distal end of each channel 115A, 115B. may be connected to a second metallic contact pad 204C, 204D. In some embodiments (not shown), distal ends of more than one channel may be connected to a single shared second metallic contact pad (204C, 204D).

In some embodiments, the membrane filter 130, the absorption layer 140. the permeable metallic electrode 150, and/or the sample collection layer 160 may each include one or more aligned dongle probe access openings 264A, 264B disposed over the metallic contact pads (for example, over the first metallic pad 204A, 204B, and the second metallic pad 204C, 204D) to enable a dongle probe to access the metallic pads. In some embodiments, the sample collection layer 160 may include additional dongle probe access openings 264C, 264D for sized and arranged to allow a dongle probe to access the permeable metallic electrode 150. In some embodiments, dimensions of each of the aligned and access openings 264A, 264B. 264C, 264D in the sample collection layer are large enough to allow access to probe heads (for example, 0.1 mm - 1 mm in diameter).

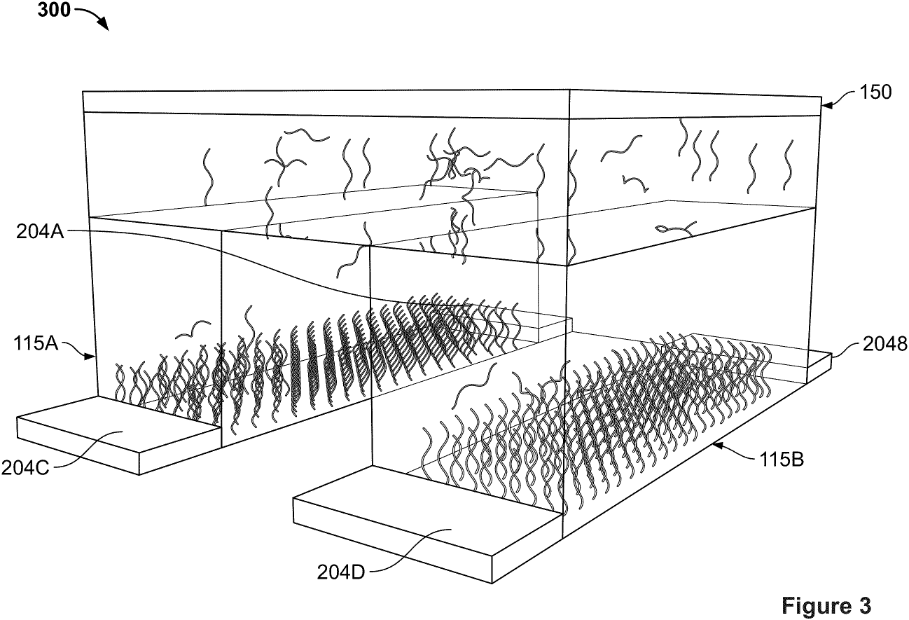

Referring to FIG. 3, a biosensor device 300 includes two biosensor channels 115A, 115B. with one biosensor channel 115A functionalized with e.g., complementary DNA, PNA, or another oligonucleotide, and used for sensing, and a second biosensor channel 115B functionalized with e.g.. non-complementary DNA, PNA or another oligonucleotide, and used as a control.

In use, a small voltage bias of about 0.01 V may be applied across both channels including the sensing channel 115A and the control channel 115B. The electrical field in each channel 115A, 115B is directed from the second metallic contact pad 204C, 204D to the first metallic contact pads 204A, 204B, respectively. In some embodiments, the second metallic contact pad 204C, 204D may be a single pad in electrical communication with both the sensor channel and control channels, while in other embodiments, two separate metallic contact pads 204C, 204D may contact the sensor and control channels 115A, 115B. A resulting current from each channel 115A and 115B (for example, a test channel current Iₜ₀, from the sensing channel 115A. and a control channel current I_{c0}) from the control channel 115B) may be measured and/or recorded.

A voltage bias (e.g., 1 V) is then applied between the permeable metallic electrode 150 and the biosensor layer 115, such that the electric field is directed from the biosensor layer 115 (that is, a positive layer, or anode) to the permeable metallic electrode 150 (that is, a negative electrode, or cathode). The electric field forces the negatively charged oligonucleotides from the saliva being tested (as well as gel-embedded nanoparticles/quantum dots, if used) to migrate to the surface of the biosensor layer 115. The electrophoresis concentrates RNA and other negative moieties at the biosensor 115 surfaces, followed by hybridization of complementary oligonucleotides to peptide nucleic acid (PNA) probes on the channels’ biosensor 115 surfaces.

The PNA probes may be attached to the channels to bind to specific analytes. In some embodiments, the nanoisland sensors are treated with PNA probes ranging from 9 base-pairs to tens of kilobase-pairs for, e.g., 10 minutes to 24 hours. The nanoisland sensors allow for robust probe attachment chemistry, direct charge transfer, and significantly increased sensor surface area. Standard thiol chemistry may be used for probe attachment. For example. 14 base pair PNA probes may be used, which are typically more stable, have a higher specificity, and exhibit faster kinetics than DNA probes, and are capable of hybridization at low ionic strength. To reduce non- specific surface binding, a PEG passivation layer may be formed on the sensor surface.

Referring still to FIG. 3,after a certain dwell/hybridization time (for example, about 1 minute), the polarity between the permeable metallic electrode 150 and the biosensor layer 115 may be reversed, resulting in a reverse migration (only for unbound or noncomplementary oligonucleotides), which removes all unbound negative species from the sensing volume. A small voltage bias of about e.g., 0.01 V is then applied across the two channels 115A, 115B and the resulting currents (that is, a testing current Iₜ₁ and a control current I_{c1}) are measured and recorded. The relative current change (before and after hybridization) in the sensor channel 115A is then subtracted from the relative current change in the control channel 115B to obtain raw data pertaining to presence and quantity of complementary oligonucleotides in the sample.

$$\text{Signal} = \frac{{\text{I}}_{\text{t0}} {\text{-I}}_{t 1}}{{\text{I}}_{\text{t0}}} \text{-} \frac{{\text{I}}_{\text{c0}} {\text{-I}}_{\text{c1}}}{{\text{I}}_{\text{c0}}}$$

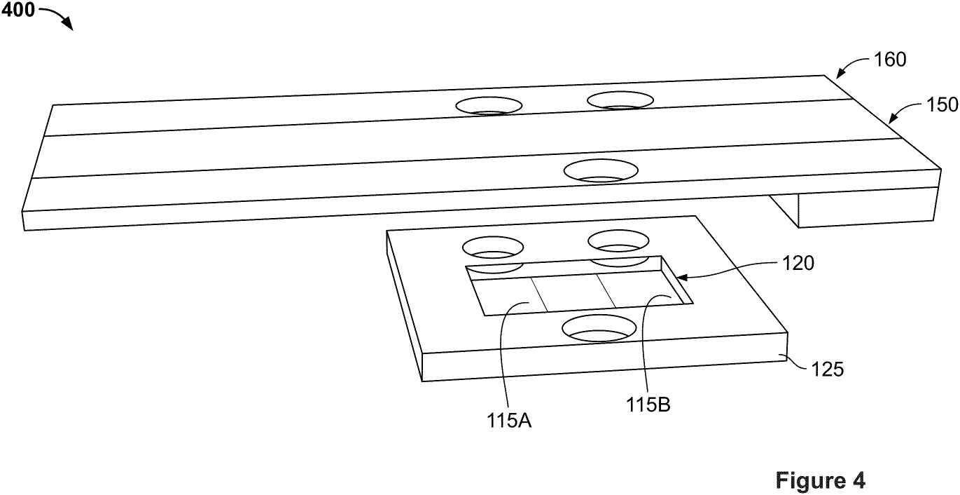

Referring to FIG. 4,in an alternative embodiment, a biosensor device 400 includes a sample collection layer 160 disposed proximate and in contact with a permeable metallic electrode 150. In this embodiment, the sample collection layer 160 is disposed under the permeable metallic electrode 150. The sample collection layer 160 may include an absorbent capillary membrane for uptake, filtration, and/or capillary transport of a sample e.g., saliva. A hydrogel layer 120, e.g., agarose, is disposed in an opening of a spacer 125, over channels 115A. 115B. A top element of the biosensor device (including the sample collection layer 160 and metallic electrode 150) may be elongated, i.e.. longer than the spacer 125 containing the hydrogel layer 120. This elongation provides case of handling, including during insertion into a dongle. In use, the permeable metallic electrode acts as a cathode, creating an electric field that pushes RNA to the channels 115A, 115B, which may include graphene-based sensors immobilized with viral-complementary DNA and a non-complementary control.

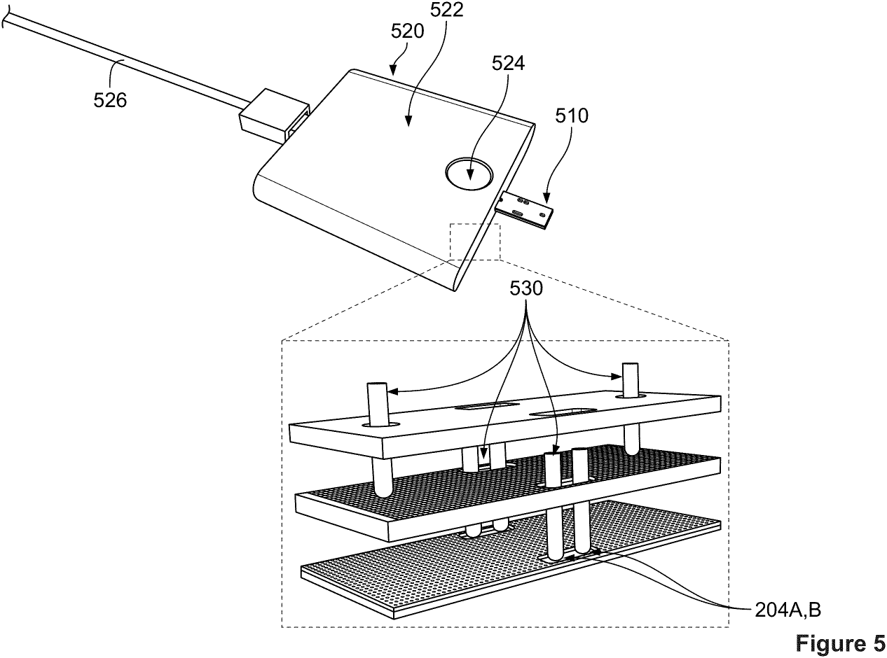

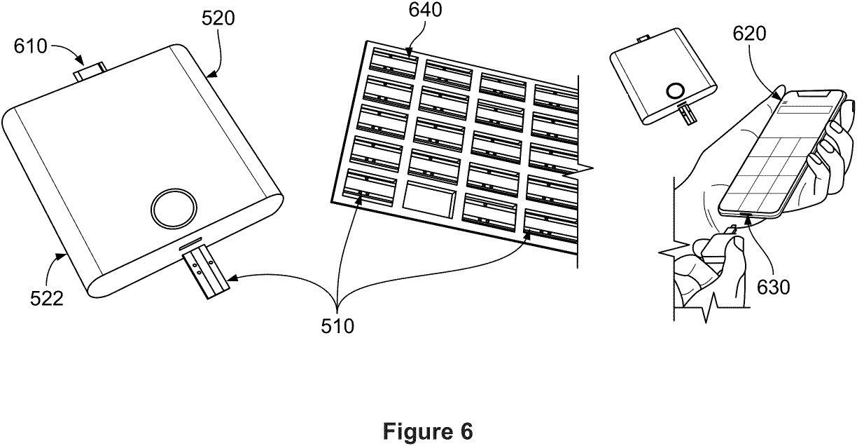

Refer to FIGS. 5 and 6, a biosensor device 510 is operatively connected to a measuring device, e.g., a dongle 520. The dongle 520 may include a housing 522. e.g., a small plastic box or other suitable box. The housing 522 may include a size ranging from 5 mm to 50 mm in each dimension. The housing 522 may house a power supply unit, a measuring unit, a mechanical chip actuation mechanism, and at least one electrode probe in electrical communication with the power supply and configured to make electrical contact to the metallic contact pads of the biosensor device 510. The mechanical chip actuation mechanism may be a spring loaded probe actuator button 524 disposed on a top surface of the housing 522, with the electrode probes being, e.g., spring-loaded electrode probes 530, configured and arranged to contact the metallic contact pads (electrodes) that are in electrical communication with the patterned biosensor layer of the biosensor device 510 when the dongle 520 is turned on via the spring loaded probe actuator button 524. In particular, the spring may be configured to, in a first position, position the at least one electrode probe through a dongle probe access opening to contact a metallic contact pad that is in electronic communication with a patterned biosensor layer, and, in a second position, to retract the at least one electrode probe.

The housing 522 may also house an interface for electrical communication with a computing device.

The dongle may be configured to connect to a cell phone or a computer via a USB port or wirelessly. Referring to FIGS. 5 and 6,the dongle 520 may be configured to connect to a communication cord/power cord 526. Accordingly, the dongle may be connected to a computer system such as a smartphone for power, communication, and/or data processing via the communication/power cord (FIG. 5). In some embodiments, the housing includes a connector 610 (FIG. 6) for plugging into a smartphone 620.

Referring to FIG. 5, the dimensions of electrode probes may be, e.g., 0.1 mm to 1 mm in diameter and 5 mm to 30 mm long. In some embodiments, when all layers are mated to each other, the electrode probes of the dongle penetrate through the openings of the device and contact their respective electrodes.

In further detail, referring to FIG. 5, when the dongle is turned on via the spring loaded probe actuator button, one or more retractable electrode probes, e.g., spring-loaded electrode probes 530, may be configured and arranged to contact the electrodes of the biosensor device 510 through one or more probe access openings extending through the sample collection layer, permeable metallic electrode, absorption layer, membrane filter, and/or spacer. In some embodiments, one or more retractable electrode probes, e.g., spring-loaded electrode probes 530, may be configured and arranged to contact the metallic contact pads through the openings in the biosensor device for providing a voltage bias across the sensor channel and/or the control channel of the biosensor layer. Furthermore, one or more additional retractable electrode probes, e.g.. spring-loaded electrode probes 530, may be configured and arranged to contact the permeable metallic electrode through the openings of the sample collection layer for providing a voltage bias between the permeable metallic electrode 150 and the biosensor layer 115 and/or to control a temperature of the device.

The measuring device, e.g.. the dongle 520, may be connected to an electrical source via a communication/power cord. It may also be connected to a computer system such as a smartphone via the communication/power cord.

Referring to FIG. 6, the dongle 520, may be configured to connect to a port 630 of a smartphone 620 directly. Accordingly, the dongle may include a connector 610 disposed on the housing 522, on a side opposite to the side that includes a reader slot configured to receive the biosensor device 510. In some embodiments, the biosensor device 510 is a sensor chip with a size comparable to that of a U.S. dime. In some embodiments, a plurality of biosensor devices 510 (or chips) may be packaged in one or more kits 640, and provided for multiple measurements.

The dongle may incorporate a mini 113 kHz sonicator board and dye against which the biosensor device may be pressed. The sonicator may assist in lysis as well enhance sample transport.

In some embodiments, the dongle 520 may include or may be replaced with another measuring device (for example, a high-throughput desktop or stand-alone device that allows simultaneous processing of multiples of chips, or a massively parallel tool for use in large laboratories or corporations).

The biosensor device described herein may reduce user workflow and enable automatic/passive sample preparation. With saliva being the analyte of choice due to the ease of collection, the described biosensor device design addresses a number of challenges, including: sample contamination with food debris, pathogens, mucus, etc. (filtration); varying chemical composition of saliva even in the same person throughout the day (deionization); low analyte concentration (preconcentration); and presence of host and pathogen DNA/RNA causing specificity issues (reverse electrophoresis).

As described above, the biosensor chip addresses these challenges utilizing the functionality of its constituent layers. For example, mechanical sample filtration is provided, by use of, e.g., a sample collection layer, e.g., a foam collection pad (with the largest pores to filter out food debris), an absorption layer (with 1-20 micron pores for filtering out mucus matrix, pathogens, and small food debris), and an electrophoresis matrix (0.1-1 micron pores to filter out cellular membrane debris).

Electrophoretic processes separate saliva constituents by charge (positive moieties migrate to the mesh electrode (cathode initially); neutral moieties do not enter the electrophoresis gel; and negative moieties, including DNA, RNA, migrate to the biosensor surface (anode initially).

Ion exchange deionization desalts saliva to <mM salt concentration, which allows for high voltage electrophoresis with rapid RNA migration, reduces Faradaic currents at electrodes, thereby providing sensor stability, and increases the ionic double layer thickness, thus increasing a sensing volume above the biosensor surface.

Preconcentration includes electrostatically concentrating DNA/RNA at the sensor surface from, e.g., a ~200 microliter sample volume to e.g.. ~50 picoliter sensing volume.

Reverse electrophoresis enhances the specificity by electrostatically removing the non-hybridized/weekly hybridized oligonucleotides from the sensing volume, with only fully hybridized moieties remaining within the sensing volume and contributing to the signal.

The biosensor device (or package) described herein may be fabricated using standard roll-to-roll and film processing practices and equipment. Suitable methods for the formation of the graphene and/or nanoislands are described in U.S. Pat. No. 9,863,885. The biosensor device provides a number of advantages. Besides improvements in sensitivity, ease of use, and sensor protection in storage, it is amenable to large, industrial-scale production of biosensors at very significant cost savings, in comparison to the semiconductor practices currently employed in sensor manufacturing. A large array of biosensors can be fabricated on planar substrate, including flexible and plastic substrates. The biosensors can be patterned on the substrate or can cover the entire surface area of the substrate.

The sensitivity of the biosensor platform described herein is based upon the well-known intrinsic sensitivity of graphene, which is further boosted by charge transfer mechanics and increased surface area originating from the noble metal nanoislands as well as from the electrophoretic preconcentration of the sample at the sensor surface.

Several mechanisms may be employed in biosensor described herein to enhance specificity enhancement, including fabrication of a second negative control channel; PEG surface passivation: short PNA probes; desalted, low ionic strength medium; elevated temperature close to PNA probe Tₘ, and reverse electrophoresis

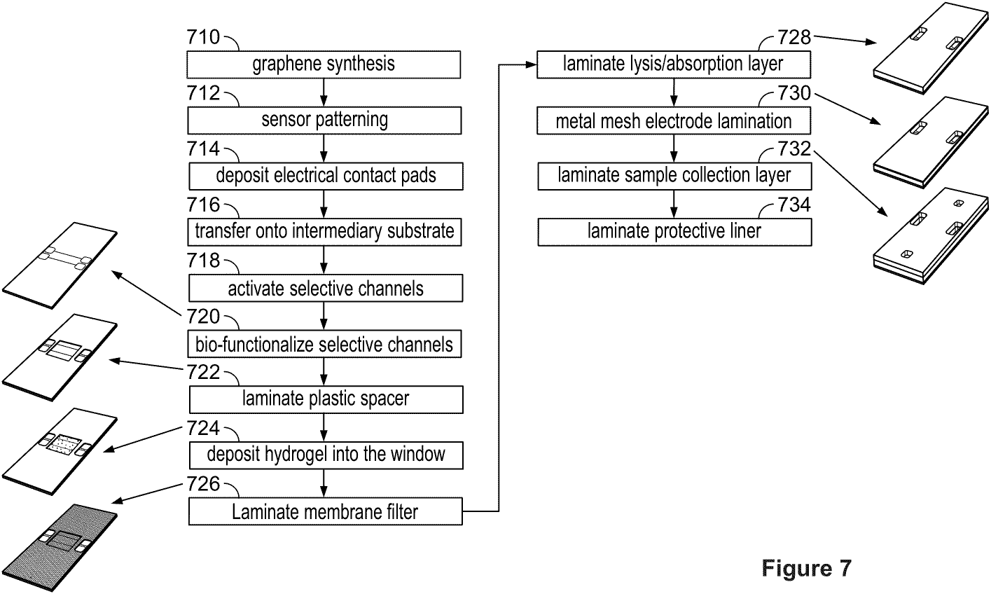

Referring to FIG. 7, a biosensor device, may be fabricated according to the following steps.

In an embodiment of a fabrication method as shown in FIG. 7, metal-assisted exfoliation (MAE) is employed. At graphene synthesis step 710, graphene may be synthesized via e.g., chemical vapor deposition (CVD) on a first substrate (for example, a metallic foil, metallic plates, wafers, or other appropriate substrate) in e.g., a batch or a continuous roll-to-roll process. Such graphene synthesis steps are known to one of skill in the art; see, e.g.. Pat. No. 9,418,839 and 9,840,024, incorporated herein by reference in their entirety.

At sensor patterning (with nanoislands and MAE layers) step 712. the material (e.g., graphene)-bearing synthesis substrate fabricated from the previous step (i.e., step 710) may be exposed to a flux of e.g., gold (or other noble metals) atoms via physical vapor deposition (PVD) (for example, thermal-, e-beam evaporation, sputtering, or other suitable deposition techniques), and masked by a shadow-evaporation mask or photolithographically patterned photoresists, in order to produce a patterned sensor layer including nanoislands of e.g., gold (or other noble metals) or MAE layers. In some embodiments, the thickness of the nanoislands ranges between 0 and 20 nm. Graphene-based nanoisland sensor (or sensing) channels may be defined in this manner. In some embodiments, the sensor (or sensing) channels may be 50 microns (i.e.. micrometers) wide and several millimeters long in a large array. For example, a 300 mm by 300 mm area of graphene can be patterned into 1250 (that is. 25 mm by 50 mm array) chips with each chip housing two individually labeled channels. In some embodiments, the dimensions of the substrate, biosensor layer, hydrogel layer, and/or spacer layer of the chips may be reduced, while dimensions of other layers (e.g.. the cover membrane filter, absorption layer, permeable metallic electrode, sample collection layer, and/or releaser liner) may be increased.

At step 714, electrical contact pads (for example: 50 nm thick gold) may be deposited on the distal or proximal ends of the sensor channels for facilitating electrical contact between the dongle probes and the channels.

At step 716, prior to transferring of the channels, a thickness of material, preferably a metal (for example, nickel, cobalt, aluminum, metal salt, or other suitable materials), may be selectively deposited above the defined channels. For example, a 50 nm thick layer of aluminum may be deposited on one channel of each chip, followed by a 50 nm thick layer of nickel deposited onto both channels of each chip. These metals in combination with the gold nanoislands may support the exfoliation of the graphene in the defined areas (non-metallized graphene areas are not removed from the growth substrate in exfoliation). In some embodiments, one channel of each chip has a nanoisland/aluminum/nickel coating, while the other channel has a nanoisland/nickel coating. This differentiation may assist in selective deprotection of individual channel sets in further steps.

At transfer step 716, after metallization, the channels may be transferred to a second (or intermediary) substrate. For example, graphene may be laminated with the second (or intermediary) substrate (for example, thermal release tape, thin-film photoresist tape, other substrates with controllable adhesion properties). The second (or intermediary) substrate is then peeled-off from the first substrate (for example, the graphene synthesis substrate) and exfoliates the areas of biosensor material (for example, graphene) that bare metallization from the first substrate. In some embodiments, the second substrate (or intermediary transfer medium) bearing the exfoliated graphene can be laminated onto a third (or final receiving) substrate directly, or it may be coated with thin films (for example, SiO₂, ceramics, or polymers) prior to the lamination. The third (or final receiving) substrate may be treated with adhesion promoters (for example, adhesives, plasma treatment, or other suitable materials), or rely purely on Van-der-Waals adhesion with the sensors. After the lamination, the second substrate (intermediary transfer medium) is removed (for example, thermally - or optically deactivated, via dissolution, or evaporated away). The third (or final receiving) substrate appears in FIGS. 1-6 as substrate 110.

At activation step 718, selective channels may be activated. In some embodiments, the graphene/nanoisland channels may be biofunctionalized selectively by de-protecting channels bearing specific coating, while retaining the protecting coatings (e.g., salt, metal, organic, or other suitable layers) on other channels. For example, the entire array of 1250 chips may be exposed to an aqueous basic solution which removes aluminum layer from the channels protected with it. The nickel-protected channels remain unaffected by the treatment.

At biofunctionalization step 720. selective channels may be biofunctionalized, chemically modified with immobilized biological molecule ligands commensurate with the detection of a specific analyte. At this step, the ligands, e.g., probes may be attached to the channels. For example, after exposing the graphene-nanoisland channel, the channel may be biofunctionalized with an analyte-specific probe (for example, antibody, oligonucleotide sequence, etc.) and/or a bio-passivation layer (for example, BSA, PEG, etc.). Further, the remaining channels may be selectively de-protected. For example, a solution of FeCl₃ (or CO gas) may remove the nickel layer while being benign to the existing biofunctionalization. After such de-protection, the bare channels may be bio-functionalized selectively.

In some embodiments, biofunctionalization is performed by depositing specific probes (e.g., PNA probes) with thiol functional groups onto the nanoislands out of solution such that the probes may self-assemble on the surface, followed by passivating the surface with PEG, BSA. and/or other molecules. Once one set of channels is biofunctionalized across the entire array, other sets of channels may be deprotected, and biofunctionalized by repeating the above process with another set of probes.

In some embodiments, more than two types of protective coatings (also referred to as capping agents) may be applied to the array to provide a multiplexed biosensor device with multiple distinctly labelled channels. For example, three types of protective coatings may be provided and selectively removed to enable functionalization of the channels with three different types of probes.

In order to be able to detect and quantify multiple analytes in a single sample, a biosensor chip preferably has multiple transducers that are individually and differently biofunctionalized. For example, a graphene-based biosensor that is capable of detecting the presence of biomolecules associated with four pathogens (e.g., influenza, coronavirus, adenovirus, and staphy lococcus aureus), has at least four channels. Each of these channels includes probes that are complementary to only one specific kind of pathogen. Additionally, specifically functionalized channels may serve as a positive or negative control channels.

Currently, to individually label select channels with specific probes, a number of serial techniques are utilized, in which individual channels are labelled either one at a time or a few at a time (e.g., MICROARRAY SPOTTER. in which probe ink microdroplets are printed on top of individual channels). Embodiments of the present invention allow large-scale fabrication of multiplexed biosensors with a massively parallel scheme for individual channel biofunctionalization. For example, the current embodiment allows to simultaneously process multiples (hundreds) of substrate sheets, each bearing tens of thousands of biosensor channels towards fabrication of about one million multiplexed biosensor chips per batch. In this example, each substrate sheet (about 300 mm by 300 mm) originally having a layer of graphene may be patterned into the arrangement of about 10,000 individual chip substrates, each including five graphene/gold nanoisland channels. Each of these five channels has a coating of one protective films (e.g.. NaCl, urea or melamine. Ni, Al, or Cu).

The purpose of the films is two-fold:
1. to protect the underlying graphene/gold nanoisland channels from the patterning/etching agents (oxygen plasma, reactive-ion etching gas, etc.)
2. to provide a mechanism for selective de-protection of the channels for biofunctionalization with target-specific probes

The capping agent film thickness is sufficiently thick to protect the underlying graphene-based channel from degradation in the graphene-etching step (e.g., using oxygen plasma or reactive-ion etching), for example 20 nm - 500 nm. The capping agent films may be deposited via physical vapor deposition techniques (e.g.. thermal evaporation or molecular beam epitaxy) through a shadow mask.

Protecting/deprotecting distinct sets of channels in the array and thus biofunctionalizing them independently provides a multiplexing capability. Accordingly, a biosensor device may be fabricated that enables detecting a number of pathogens/analytes in a sample simultaneously, e.g., COVID-19, H1N1, H1N3, COVID-19 delta, and have a negative control channel.

For example, in an embodiment, five different chemistries may enable fabricating five individually labelled channels per chip. Further, by applying pick-and-place robotics, one may multiplex to virtually unlimited number of analytes in multiples of 5.

In an exemplary process, graphene/nanoisland material may be patterned into five channels per chip with each channel protected by the following capping agents:
NaCl (removable by water)
urea or melamine (removable by isopropyl alcohol)
Al (removable by NaOH)
Ni (removable by CO gas)
Cu (removable by FeCl₃ solution)
Each capping layer material in the set is preferably selected such that it can be removed in the de-protection step using a chemical solvent or a process to which all of the other capping agents are impervious. The exposure of the array to the de-protecting solvents/processes proceeds in an order such that only channels protected by one capping agent are de-protected at a time. For example, considering the set of channels protected by the abovementioned capping agents (NaCl, urea or melamine. Al, Ni, Cu), initial exposure to water simultaneously would remove NaCl and urea, thus de-protecting two sets of channels and should be avoided. Instead, the array is preferably initially exposed to an alcohol, that removes urea and leaves the other sets of channels unaffected. Water exposure is preferably the second de-protecting solvent exposure.

These channels may then be processed in the following order. First, the urea capping layer may be removed by immersing the array in or rinsing with alcohol, followed by biofunctionalization of that de-protected channel with ligands such as probes and passivation chemistry in alcoholic solutions of these probes and passivation chemistries.

Second, the NaCl capping layer is removed by water followed by biofunctionalization of that channel with ligands and passivation chemistry in alcoholic or aqueous solutions of these probes and passivation chemistries.

Third, the Al capping layer is removed by NaOH followed by biofunctionalization of that channel with probes and passivation chemistry in alcoholic or aqueous solutions of these probes and passivation chemistries. Fourth, the Ni capping layer is removed by CO gas followed by biofunctionalization of that channel with probes and passivation chemistry in alcoholic or aqueous solutions of these probes and passivation chemistries. Fifth step, the Cu capping layer is removed by FeCl₃ solution followed by biofunctionalization of that channel with probes and passivation chemistry in alcoholic or aqueous solutions of these probes and passivation chemistries.

These chemical reactions may be performed in large baths on multiple, e.g., 100 array sheets, with each sheet being, e.g., 1 foot by 1 foot, such that each step processes up to 1 million channels. After five steps, up to 1 million chips may be ready for additional functional layer lamination, dicing, sterilization and packaging.

At plastic spacer lamination step 722, a plastic spacer may be laminated. In some embodiments, a plastic sheet containing an array of 1250 spacers (e.g., 5 mm by 12 mm) and an adhesive layer is further laminated onto the sensor array such that the small perforations in the spacers are overlaid with the gold contact pads and the window for subsequent gel deposition is overlaid above the sensor channels.

At hydrogel deposition step 724. a hydrogel layer, e.g., a thin layer of agarose with desired chemical composition and physical properties may be deposited into a window in the plastic spacer (or a chip spacer window). The hydrogel can either cover the entire surface of the substrate or can be patterned to cover selected areas using doctor-blading, slot-die coating or other film coating techniques. For example, a pressure sensitive adhesive film of a given thickness (e.g., silicone-based adhesive with a thickness of 100 microns) with a removable liner and cut out openings can be laminated over the substrate such that the biosensors are disposed in the cut out opening of the film. Next, agarose solution is doctor-bladed into the cut-out openings of the adhesive film and forms a hydrogel. The adhesive film release liner can then be removed exposing the top side of the adhesive. As indicated below, in further steps, filtration/absorption medium film can be laminated over the stack such that it makes direct contact with the hydrogel and the adhesive and is thus secured in place.

In some embodiments, electrophoretic gel medium precursors are deposited into the chip spacer windows via e.g., doctor-blading, where the thickness of the spacer determines the resulting gel thickness.

At membrane filter lamination step 726, a track-etched membrane filter may be laminated.

At lysis/absorption layer lamination step 728. the lysis/absorption layer may be laminated. In some embodiments, a porous layer, e.g., a cellulosic matrix containing lysis-assisting compounds and probe-access perforations is laminated onto the chip array assembly.

At metal mesh electrode lamination step 730, a metal mesh electrode may be laminated. A perforated thin-metal mesh film (e.g., foil) with probe-access openings is laminated onto the chip array assembly. It can be adhered to the filtration/absorptive medium film with adhesives or physically (for example, by press-rolling the expanded metal mesh over the filter/absorption film).

At sample collection layer lamination step 732, a sample collection layer may be laminated. In some embodiments, a sample collection layer (e.g., a reticulated polyurethane film) with probe-access perforations is laminated onto the chip array assembly.

At protective liner lamination step 734, a removable protective liner may be laminated. In some embodiments, a continuous protective liner (e.g., PET) film is laminated onto the chip array assembly. The protective liner helps protect the biosensor device from evaporation and contamination in post-processing and storage.

At protective liner lamination step 734, the assembled array may be diced into individual chips via e.g., laser or roller-blade cutting. In some embodiments, the assembled array is further sterilized and packaged for individual use. Accordingly, a single large area sheet of the described composite biosensor package may contain an array of thousands of individual biosensors.

In alternative embodiment (not shown), graphene may be synthesized as described above with respect to step 710.

In a next step, the graphene may be transferred onto a final receiving substrate. In some embodiments, traditional polymer-supported wet- or dry- graphene transfer techniques may be used to coat the third (or final receiving) substrate with a layer of graphene.

In a next step, lithography of the graphene channels may be conducted. In some embodiments, traditional photolithography may be used to pattern the graphene into defined sensor channels

In a next step, the electric contact pads may be deposited. Electrical contact pads are deposited (e.g., 50 nm-thick gold) on the distal ends of sensor channels to facilitate electrical contact between the dongle probes and the channels.

In a next step, optional nanoislands may be deposited, as described above. Subsequently, selected channels may be activated and overlying layers formed, as described above.

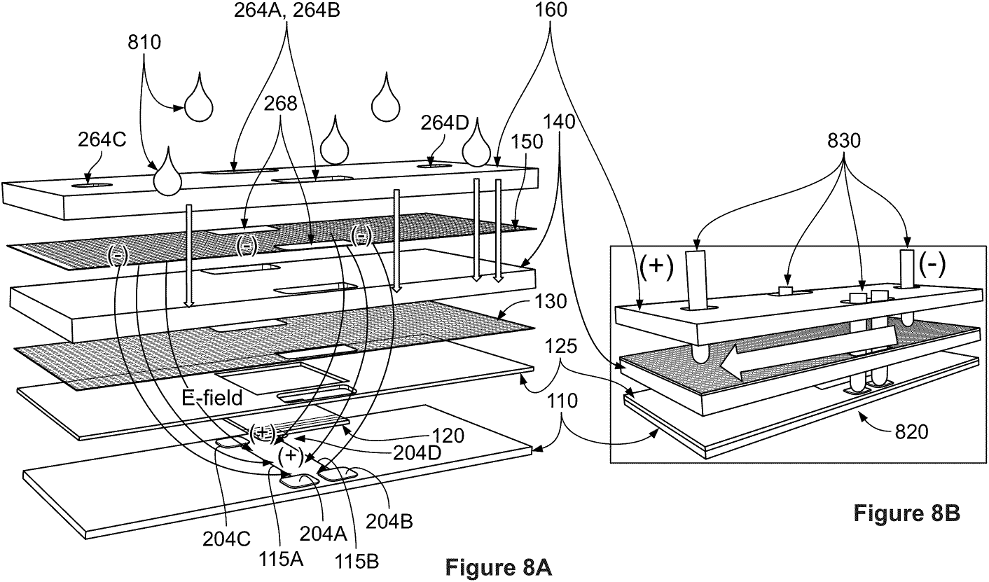

FIGS. 8A and 8B illustrate the method of use and/or how the device works or functions

Referring to FIG. 8A,in use, the absorption layer 140 may absorb desalted saliva, and provide the lysis chemistry. The cathode in forward electrophoresis moves and concentrates the target at the sensor surface, while the anode in reverse electrophoresis removes the unbound oligonucleotides from the sensing volume. In some embodiments, the first metallic contact pad 204A, 204B works as a source electrode, and the second metallic contact pad 204C, 204D works as a drain electrode, while in other embodiments, the first metallic contact pad 204A, 204B works as a drain electrode, and the second metallic contact pad 204C, 204D works as a source electrode.

A saliva sample may be analyzed as follows. Saliva 810 may be absorbed by the sample collection layer 160 and gets deionized, with ion exchange deionization in e.g.. a deionization matrix, desalting saliva to <1 mM salt concentration. The permeable metallic electrode may be heated, e.g.. by Joule heating, by applying about 10 V voltage (about 2.5 A current from e.g., the dongle) to the permeable metallic electrode with dongle probes. The sample, e.g., saliva, may be heated to about 95° C. to assist with lysis. In some embodiments, the absorption layer may include pre-deposited lysis chemistry (for example, guanidinium thiocyanate (GITC), suitable surfactants) that further assists in lysis of the sample.

A voltage bias of about 0.01 V may be applied across the biosensor layer including two sensing channels (that is, the sensor channel 115A and the control channel 115B), and a resulting current from each channel (for example, a test channel current Iₜ₀ from a test channel, and a control channel current I_{c0} from a control channel) may be measured and/or recorded.

Referring still to FIG. 8A, in the next step, a voltage bias (for example. 1 V) is introduced between the permeable metallic electrode 150 and the biosensor layer 115, through the absorption layer 140, the membrane filter 130, the spacer 125, and the hydrogel layer 120. In some embodiments, the electric field is directed from the biosensor layer 115A, 115B (that is, a positive layer, anode) to the permeable metallic electrode 150 (that is, a negative electrode, or cathode). The electric field forces the negatively charges (for example, oligonucleotides) to migrate to the surface of the biosensor layers (or sensing channels) 115A. 115B, which are positively charged. This electrophoresis concentrates RNA and other negative moieties at the biosensor surfaces, followed by hybridization of complementary oligonucleotides to PNA or other oligonucleotide probes on the biosensor surfaces. The PNA probes may be synthesized to target oligonucleotides of interest such as single stranded RNA, DNA, and/or double stranded DNA and RNA. Other oligonucleotide probes may be designed to selectively bind various types of targets. For example, DNA oligomer probes may be designed to bind peptides and/or proteins (e.g., antibodies, enzymes, etc.).

In use, the retractable electrode probes may make electrical contacts with a biosensor device 810, one or more optical elements (for example, light source, mirrors, photodetectors, and/or other suitable elements) for e.g.. optical sensor interrogation, and/or supporting electronics (for example, power source-meter units, communication units, and/or other suitable electronics).

After a certain dwell/hybridization time (for example, about 1 minute), the polarity between the permeable metallic electrode and the biosensor layer is reversed, resulting in a reverse migration of unbound, noncomplementary oligonucleotides, which removes all unbound negative species from the sensing volume directly above the channels. A small voltage bias of about e.g., 0.01 V is then applied across the two sensing channels and the resulting currents (for example, a testing current Iₜ₁ and a control current I_{c1}) are measured and recorded. The relative current change before and after hybridization in the sensor (or test) channel (that is. Iₜ₀ and Iₜ₁) is then subtracted from the relative current change in the control channel (that is. I_{c0} and I_{c1}) to obtain raw data pertaining to presence and quantity of complementary oligonucleotides in the sample, which is represented by:

$$\text{Signal} = \frac{{\text{I}}_{\text{t0}} {\text{-I}}_{t 1}}{{\text{I}}_{\text{t0}}} \text{-} \frac{{\text{I}}_{\text{c0}} {\text{-I}}_{\text{c1}}}{{\text{I}}_{\text{c0}}}$$

Referring to FIG. 8Ain use, the hydrogel layer 120 may keep immobilized bio-ligands on top of the biosensor layer 115A, 115B hydrated and maintains the required salinity and pH for prolonged periods of time in storage. In some embodiments, the hydrogel layer 120 may also serve as a medium for electrophoretic separation of bioanalytes. The hydrogel layer 120 may be selected to have a desired porosity, salinity, pH. and precharged with surfactants and other desired compounds.

Referring still to FIG. 8A, in use, the absorption layer 140, which is disposed above the membrane filter 130 and the hydrogel layer 120, absorbs a volume of saliva upon its application to a biosensor device (for example, via licking) through the openings 268 in the permeable metallic electrode 150.

The absorption layer 140 may include a material selected such as to be able to absorb a volume of saliva that is comparable to the volume occupied by the material itself. In addition to being a receptacle for a collected saliva sample, the absorption layer 140 may filter out large particles (for example, food and cell debris, large pathogens, and/or other particles) that remain on the surface of the absorption layer 140.

The permeable metallic electrode 150 may serve as a capping support to the absorption layer 140, as well as an electrode in electrophoresis steps.

Referring to FIG. 8B, when the probes 830 are connected to the biosensor device 820 (or the chip), a vertical voltage bias (for example. 1 V) may be applied between the permeable metallic electrode and the biosensor layer, and a small horizontal voltage bias (for example, 0.01 V) may be applied across the sensing channel and the control channel. When the vertical voltage is applied, the deionized saliva may be squeezed out of the sample collection layer through the permeable metallic electrode and is absorbed (or lysed) by the absorption layer. The electric field directed from the biosensor layer (that is, a positive layer, or anode) to the permeable metallic electrode (that is, a negative electrode, or cathode) may force the negatively charged oligonucleotides from the saliva being tested (as well as gel-embedded nanoparticles/quantum dots, if used) to migrate to the surface of the biosensor layer. The electrophoresis concentrates RNA and other negative moieties at the biosensor surfaces, followed by hybridization of complementary oligonucleotides to peptide nucleic acid (PNA) probes on the channels’ biosensor surfaces. When the horizontal voltage is applied, the electrical field in each channel is directed from the second metallic contact pad to the first metallic contact pads, respectively, as described above in FIG. 2.

In some embodiments, the permeable metallic electrode 150 heats a sample to a required temperature (e.g., 60 - 95° C. ) for lysis within e.g., a few seconds, and maintains the required temperature just below the probe oligonucleotide specific melting point (e.g., 20 - 85° C.) throughout the measurement. In some embodiments, the permeable metallic electrode 150 may heat a sample to at least about 120° C. for decontamination.

Referring still to FIGS. 8A and 8B, in some embodiments, upon application of the saliva to the surface of the biosensor device and its absorption by the absorption medium, the device can be electrically addressed (for example with retractable spring-loaded electrode probes) via the metal electrode pads deposited onto the sensor edges as well as via the metallic perforated foil. Electrically biasing the sensor is required for obtaining a signal associated with the presence and quantity of a given analyte in the sample. This is accomplished by monitoring the change in the electrical characteristics of the sensor associated with the change in the chemical composition of the medium surrounding the sensor. For example, binding of an analyte complementary to the immobilized ligand on the sensor can induce a direct charge transfer to the sensor and thus modify the conductance of the sensor channel (alternatively, even if no direct charge transfer occurs upon the binding event, the conjugation of the complementary analyte modifies the chemical and physical properties of the medium directly surrounding the sensor channel (index of refraction, charge distribution, local pH. etc.). This change can be investigated electronically, optically, or using an optoelectronic combination of investigatory methods. Regardless of the sensing method, the following description of the electrophoretic functions of the biosensor device are applicable.

Creating an electrical bias between the sensor and the perforated metal foil creates an electric field that induces an electrostatic force onto charged biomolecules and particles. This forces them to migrate according to their charge (electrophoresis) - negative molecules towards the anode, and positive - towards the cathode. This way, molecules with a given charge can be concentrated at the sensor surface in concentrations greatly surpassing their initial concentration in the sample. For example, in order to detect viral RNA in patient’s saliva for viral disease diagnosis, the device can be energized to create an electrical bias between the cathode (perforated metal foil) and the anode (sensor). The negatively charged RNA molecules will migrate to the sensor and create a zone of increased concentration at the sensor surface. If the sensor surface has immobilized complementary oligonucleotides (probes), the RNA or DNA chains will bind to these probes. The binding can be detected optically/electronically. Additionally, by reversing the polarity on the electrodes, the unbound non-complementary oligonucleotides can be removed from the sensor surface for added sensor sensitivity.

The biosensor device architecture described herein includes a biosensor on a substrate capped with a thin layer of hydrogel, an absorbent filter medium, and a permeable (perforated) metal foil. This architecture allows salivary sample collection, filtration, and electrophoresis, as well as enables prolonged sensor storage.

The hydrogel keeps the immobilized bio-ligands on top of the biosensors hydrated and maintains the required salinity and pH for prolonged periods of time in storage. It also serves as a medium for electrophoretic separation of the bioanalytes. The hydrogel layer may be selected to have a desired porosity, salinity, and pH, and may be precharged with surfactants and other desired compounds.

The sample collection layer absorbs a volume of saliva upon its application to the device (via licking, for example). The absorbent material is selected to be able to absorb a volume of saliva comparable to the volume occupied by the material itself. In addition to being a receptacle for the collected saliva sample, the absorbent medium filters out large particles (food and cell debris, large pathogens) that remain on the surface of this medium.

The perforated metal sheet may serve as a support to the absorbing medium as well as an electrode in electrophoresis steps.

Upon application of the saliva to the surface of the biosensor device and its absorption by the sample collection layer, the biosensor device may be electrically addressed (for example with retractable spring-loaded electrode probes) via the metal electrode pads deposited onto the sensor edges as well as via the metallic perforated foil. Electrically biasing the biosensor enables the obtainment of a signal associated with the presence and quantity of a given analyte in the sample. This is accomplished by monitoring the change in the electrical characteristics of the sensor associated with the change in the chemical composition of the medium surrounding the sensor. For example, binding of an analyte complementary to the immobilized bio-ligand on the biosensor can induce a direct charge transfer to the biosensor and thus modify the conductance of the sensor channel. Alternatively, even if no direct charge transfer occurs upon the binding event, the conjugation of the complementary analyte modifies the chemical and physical properties of the medium directly surrounding the biosensor channel (index of refraction, charge distribution, local pH, etc.). This change may be investigated electronically, optically, or via an optoelectronic combination of investigatory methods. Regardless of the sensing method, the following description of the electrophoretic functions of the sensor package is applicable.

Creating an electrical bias between the biosensor and the perforated metal foil creates an electric field that induces an electrostatic force onto charged biomolecules and particles. This forces the charged biomolecules and particles to migrate according to their charge (electrophoresis) -negative molecules towards the anode, and positive molecules towards the cathode. This way, molecules with a given charge can be concentrated at the sensor surface in concentrations greatly surpassing their initial concentration in the sample. For example, in order to detect viral RNA in patient’s saliva for viral disease diagnosis, the device can be energized to create an electrical bias between the cathode (perforated metal foil) and the anode (biosensor). The negatively charged RNA molecules migrate to the biosensor and create a zone of increased concentration at the biosensor surface. If the biosensor surface has complementary oligonucleotides (probes) immobilized thereon, the RNA or DNA chains bind to these probes. The binding can be detected optically/electronically. Additionally, by reversing the polarity on the electrodes, the unbound non-complementary oligonucleotides may be removed from the biosensor surface for added sensor sensitivity. Electrophoresis allows the separation of saliva constituents by charge (positive moieties migrate to the mesh electrode (cathode initially); neutral moieties do not enter the electrophoresis gel; negative moieties, including DNA, RNA migrate to the biosensor surface (anode initially).

A reader facilitates the use of the biosensor device. For example, the reader may be a smartphone dongle adapted to accommodate individual biosensor devices. The dongle may house retractable electrode probes (that make electrical contacts with the biosensor device elements) and optical elements (light source, mirrors, photodetectors, etc.) for optical sensor interrogation as well as required supporting electronics (power source-meter units, communication units, etc.) The dongle may be connected to a smartphone for power/communication/data processing.

In use, a user may employ the described biosensor device to, e.g.. detect the presence of a viral agent in one’s saliva and thus aid in diagnosing the viral disease. The following components are needed for such analysis: the biosensor device described herein, a measuring device (for example a dongle), and a computing device (for example, a smartphone with appropriate software).

An algorithm for using the biosensor device may be as follows.
1. An individual biosensor device is removed from its storage container.
2. The protective release liner is removed from the top surface of the biosensor device.
3. The saliva is deposited on top of the biosensor device (for example, by licking the top surface of the biosensor device). A volume of the saliva is absorbed into the filtration/absorptive medium.
4. The biosensor device is then inserted into the measuring device (for example, a dongle, which may be in communication by USB or wirelessly with a smartphone connection). The measuring device is activated (e.g., engaging the biosensor via retractable electrical probes for electronic sensor interrogation, or via illuminating the biosensor with a light source in the optical interrogation mode).
5. Data from the biosensor device is then transmitted to the smartphone and analyzed using dedicated software.

### EXAMPLES

### Example 1 - Work Flow

A minimal configuration for a platform including a biosensor device is a personal home-based screening tool, consisting of a cellphone-tied or stand-alone reader and inexpensive disposable chips (see FIG. 6). The workflow is minimal and is reduced to the following steps: unpacking a biosensor device (or chip), depositing saliva via active drooling onto the biosensor chip by depressing the sample collection layer (for example, a polyurethane foam strip) with the tongue several times, inserting the chip into a reader slot, and pressing the button on the reader

In some embodiments, a minimized workflow includes required sample preparation steps, including saliva collection, filtration, desalting, lysis, electrophoretic separation and electrostatic concentration, as well as sensing proper.

An operation of the chip may be described as a “vertical flow assay” - the sample, e.g., saliva, is modified and processed as it passes vertically across the layer stack driven via mechanical, capillary, and electroosmotic forces. The individual layers may be hundreds of microns thick, so the sample transport (including electrophoresis) is fast, resulting in sample-to-answer in under, e.g., 120 seconds.

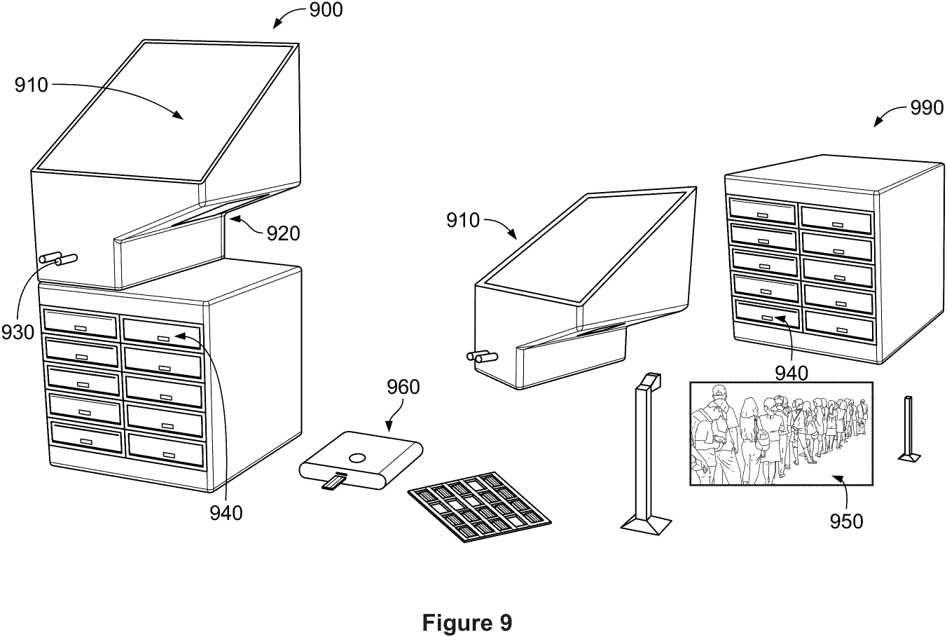

Referring to FIG. 9, a minimized workflow allows the use of biosensor technology in environments where en masse screening may be required, for example airline boarding prescreening. The individual barcoded sensor chips may be associated with a personal identification/boarding pass via a user interface scanner 920. The chip dispensing and personal identification may be decoupled from the sample analysis in space and time to allow a high throughput screening of crowds at a rate of, e.g., 12 seconds per person. In particular, a point of entry screening unit 900 may include a touchscreen user interface 910, an ID/boarding pass scanner 920. a chip dispensing element 930, and a number of, e.g., ten, swappable bays 940 for reading chips. In an example of screening process 990, the user 950 may take the barcoded sensor chip, peel the liner, and lick the strip. The chip may then be inserted into an available reader slot. The reader may associate the chip ID with a person and result via the barcode. The result may be quickly sent to the boarding crew, e.g., in less than two minutes. Upon scanning of an ID and/or boarding pass, a chip may be dispensed, with a barcode associated with the ID and/or boarding pass. A reader with a positive result may get swapped with a new reader, with the used reader being sent for decontamination.

Accordingly, a platform may include the three parts: a disposable chip (i.e., a biosensor device as described above), a reader (i.e., a dongle as described above), and processing/user interface software. The reader may be wirelessly connected to a cellular phone or a cloud-based user interface. The disposable chip may be a e.g., 0.5″x1″x0.1″ sterilized biosensor device.

Saliva collection may be performed as follows. A user may remove an individual biosensor chip out of the package (e.g., a moisture controlled “blister pack”), peel away the protective liner (e.g., attached on a handling side of the chip), hold the chip by the handling side and with the foam pad facing downwards, press his tongue against the foam pad several times, until the foam feels wet (moisture/temperature indicating dye may be incorporated for compliance control), and fold the peeled protective liner back into its original position to cover the biosensor chip.

The user 950 may then insert the chip into the reader slot, press the button to engage the reader, and leave the chip/ in the reader for about 2 minutes until the reading is complete. The progress may be monitored on the user interface screen (cell phone/computer) or via a blinking light and/or chime tones on the dongle. The test results may be displayed on the user interface screen as a “green” NEGATIVE or “red” POSITIVE indicator. If a positive or otherwise inconclusive result is obtained, a repeat measurement may be suggested (after test chip deactivation and reader slot decontamination).

Regarding disposal, in a home setting, the chips may be disposed of in household trash bins, whereas in the point-of-care setting, the chips may be disposed of in standard biohazard boxes. A chip with a positive reading may be automatically thermally deactivated in the dongle (using the internal heating element) by, e.g., heating the chip to 120° C. for two minutes. After deactivation, the chip can be disposed of without additional precautions.

As a screening tool, the dongle may need to be decontaminated after a reported positive result to avoid repeated false positives. For decontamination, a disinfectant- soaked foam pad may be inserted into the dongle and the dongle button pressed several times, mechanically cleaning the chip chamber and the electrodes with disinfectant. The decontamination chip may remain inserted for a sufficient period, e.g., 30 minutes, after which the rinsing and drying chips are used in succession.

### Example 2 Sensing Principles

### Amperometry (2-Electrode Mode)

The extreme sensitivity of graphene to changes in a local chemical environment stems from the fact that the graphene is a 2D conductor material (e.g., one atom thick). Thus, the entirety of the electron cloud of the sensor strip may be available to interact with the environment. Additionally, extremely high electron mobility in graphene means that any perturbation to the electron cloud gets translated into a detectable electronic signal (e.g., change in the electrical current running across the sensor strip). For example, in the event of an oligonucleotide chain from the sample solution hybridizing to the complementary probe that is conjugated to the sensor, the additional charge from the hybridized DNA or RNA changes the capacitance of the system, which modulates the current.

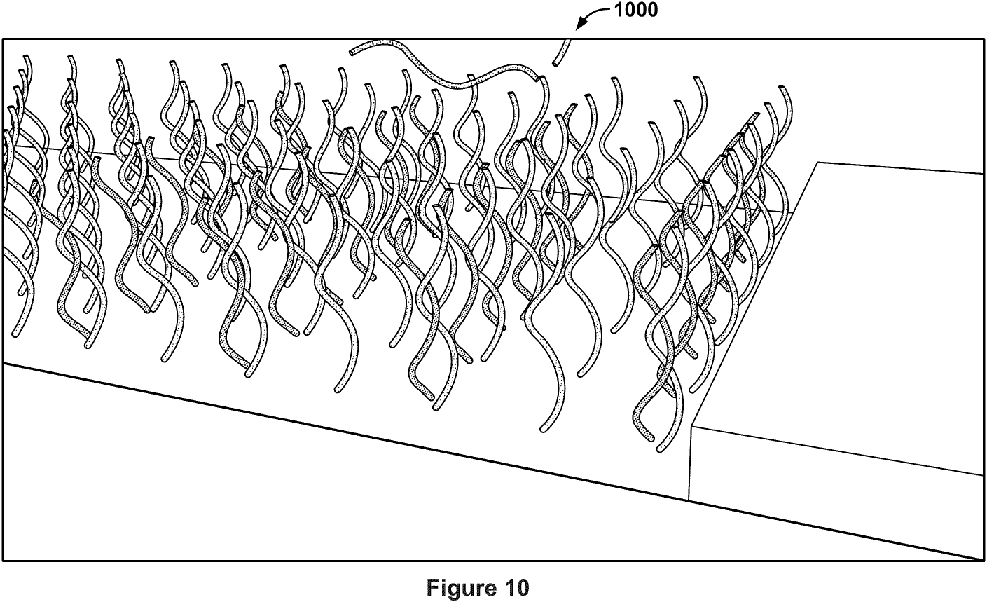

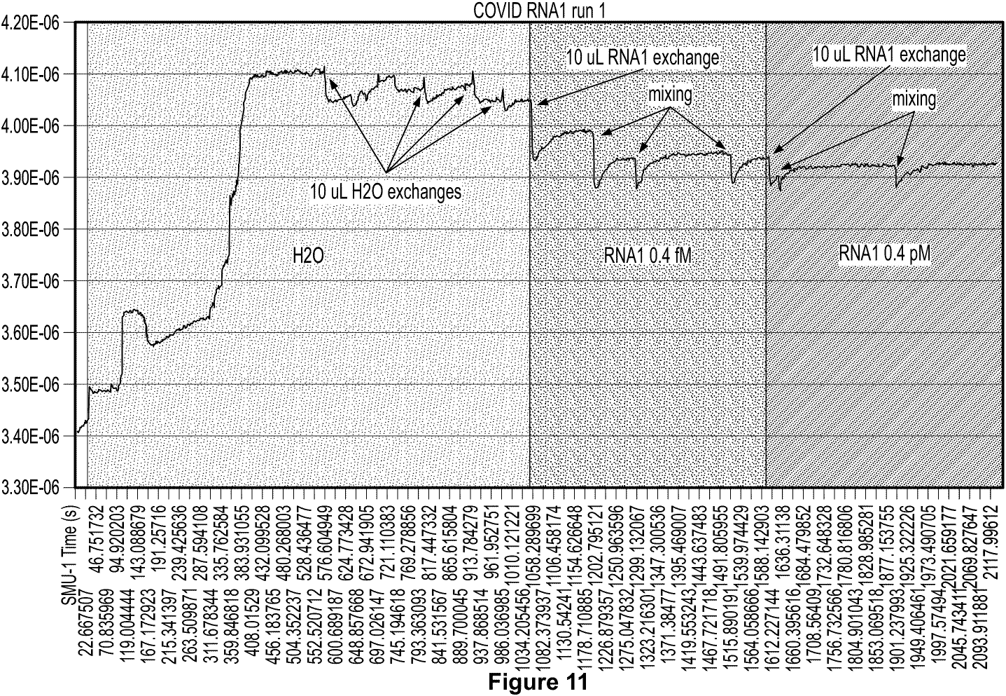

Referring to FIG. 10, complementary RNA is hybridized to immobilized DNA in a biosensor device 1000 in accordance with an embodiment of the invention, and FIG. 11 is a graph illustrating the resulting electrical signal. In particular, one mode of operation of the sensors is presented in FIG. 11, which is a plot of monitored current against time as buffers of varying COVID RNA fragment concentration (0, 0.4 aM. 4 aM. 0.4 fM, and 0.4 pM) are introduced to the sensor with complementary DNA probes. It can be seen that all liquid handling events (blank buffer exchange or spiked buffer introduction) are reflected in the sensor current as they disturb the equilibrium capacitance of the system. Although, in the case of blank buffers, within seconds the current is returned to the original baseline, whereas in the presence of hybridizable moieties even in low concentrations a new baseline is established. The difference between the stable current baselines represents the monitored signal that indicates the presence (and quantity - up to picomolar concentrations) of the oligonucleotide of interest.

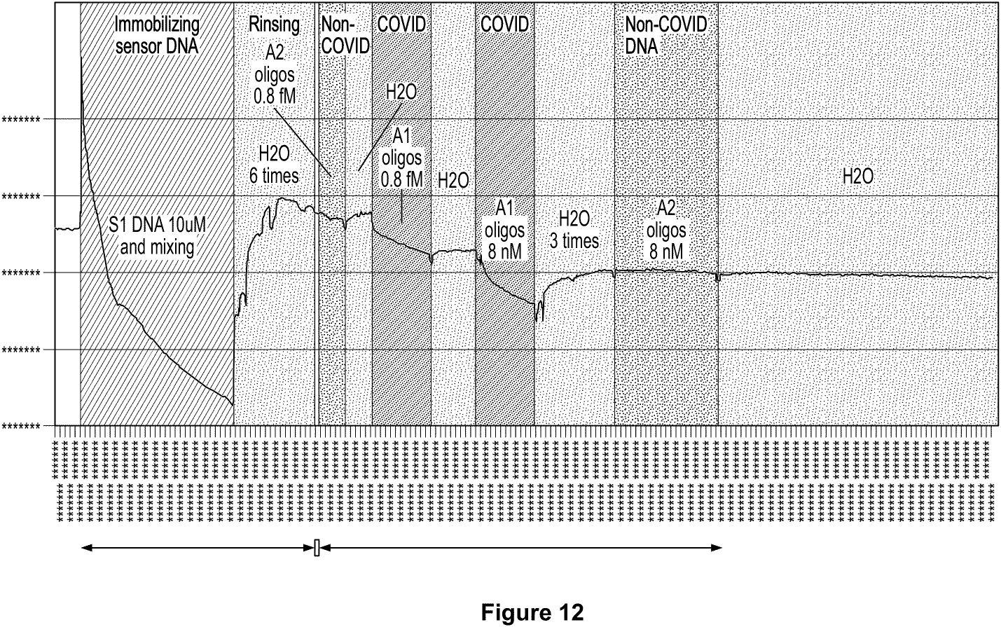

FIG. 12 illustrates the specificity of the signal to only hybridizable sequences. Nonspecific oligonucleotides only very slightly and transiently modulate the signal. To increase the specificity and signal to noise ratio, biosensor devices described herein typically have two sensors - a sensing channel, with probes complementary to the target, and a negative control channel, with probes of the same length, yet not complementary to known sequences. The chip may be designed such that both channels are exposed simultaneously to the same sample fraction and thus nonspecific events can be excised from the data stream. Importantly, the same operation principle is applicable irrespective of the assay type - molecular or antigen. By conjugating antibodies to the sensors, specific antigen presence can be monitored and quantified.

### Dirac Point Monitoring (3-Electrode Mode)

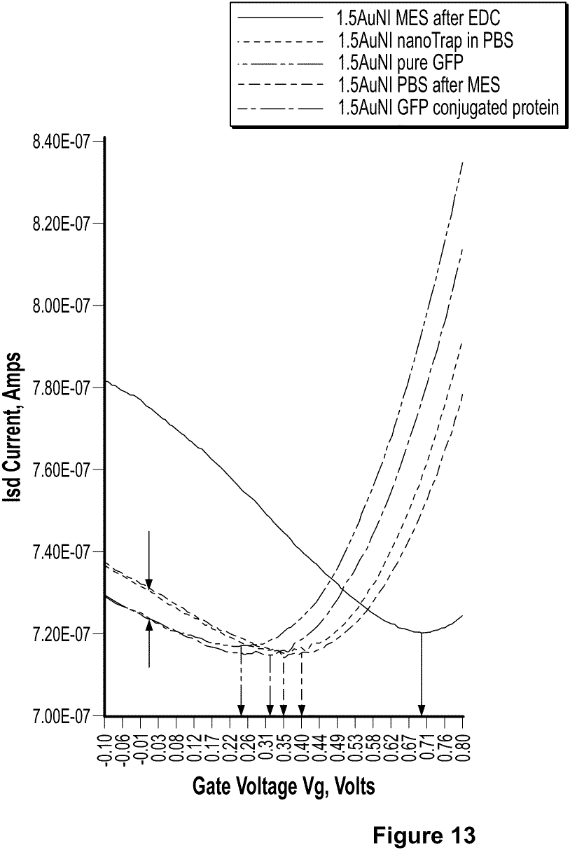

Referring to FIG. 13, another, more conventional mode of operation of the graphene-based biosensors includes identification of hybridization events via a shift the Dirac point, i.e., the point of minimal current as the gate voltage is scanned. Shifts of the Dirac point originate from the same phenomena as described before - chemical environment change. The biosensor devices described herein are extremely sensitive in the real-time amperometric (2-electrode) mode of operation, making the Dirac point monitoring approach obsolete (yet available).

### Optical Mode of Operation

The sensor material described above (graphene/noble metal nanoislands) may be plasmonically active and used in traditional SPR/LSPR, and/or Raman modes. In conjunction with the lenticular (replacing the need for a prism) transparent chip substrate film, this makes optical interrogation of the biosensor chip available through the bottom substrate. Employing both electronic and optical investigation in a biosensor may be a useful and powerful feature in some of the more challenging assays.

The use of the biosensor device described herein is not limited to analysis of saliva and other medical diagnostics. Besides medical diagnostics, the biosensor described herein may be used in agriculture (e.g., analysis of crop health), animal husbandry, fishing industries (e.g., determining the presence of pathogens in food stock) and many other use cases that require identification of biological molecules.

While the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements, the present invention being limited only by the claims appended hereto and the equivalents thereof.

What is claimed is:

1. A biosensor device for detecting an analyte in a sample, the biosensor device comprising:
a vertical stack comprising:
a patterned biosensor layer;
a hydrogel layer disposed above and in contact with the patterned biosensor layer,
a permeable metallic electrode disposed above the hydrogel layer, and
a sample collection layer disposed proximate and in contact with the permeable metallic electrode.

2. The biosensor device of claim 1, wherein the patterned biosensor layer comprises at least one of a nanocarbon material, graphene with noble metal nanoislands formed thereon, a transition-metal dichalcongenide (TMD), a two-dimensional (2D) material, or a one-dimensional material.

3. The biosensor device of claim 1, wherein the hydrogel layer comprises at least one of agarose or polyacrylamide.

4. The biosensor device of claim 1, wherein the hydrogel layer is adapted to electrophoretically filter and separate a plurality of sample moieties by size.

5. The biosensor device of claim 1, wherein the hydrogel layer is adapted to electrophoretically concentrate a plurality of oligonucleotides from the sample at a surface of the patterned biosensor layer.

6. The biosensor device of claim 1, wherein the permeable metallic electrode is adapted to control a temperature of the biosensor device.

7. The biosensor device of claim 6, wherein the temperature results in lysis of the sample.

8. The biosensor device of claim 7, wherein the temperature enhances hybridization of the analyte to a biomolecular ligand immobilized in contact with the patterned biosensor layer.

9. The biosensor device of claim 1, wherein the permeable metallic electrode is configured to act as at least one of a cathode or an anode during electrophoresis through the hydrogel layer.

10. The biosensor device of claim 1, wherein the patterned biosensor layer is disposed above and in contact with a substrate.

11. The biosensor device of claim 10, wherein the substrate comprises at least one of glass, a polymeric film, a single crystal material, surface-enhanced Raman Spectroscopy (SERS) substrate, a localized surface plasma resonance (LSPR) substrate, a surface plasma resonance (SPR) substrate, fluorescence in situ hybridization (FISH) labeled substrate, or an electrochemical sensing substrate.

12. The biosensor device of claim 1, further comprising a biomolecular ligand immobilized over and in contact with the patterned biosensor layer.

13. The biosensor device of claim 12, wherein the biomolecular ligand comprises at least one of a plurality of oligonucleotide probes, antibodies, antigens, or enzymes.

14. The biosensor device of claim 1, wherein the sample collection layer comprises an absorbent filter medium.

15. The biosensor device of claim 1, wherein the sample collection layer comprises a matrix.

16. The biosensor device of claim 1, wherein the sample collection layer is disposed above the permeable metallic electrode.

17. The biosensor device of claim 1, further comprising an absorption layer disposed between the hydrogel layer and the permeable metallic electrode.

18. A dongle in electrical communication with the biosensor device of claim 1, the dongle comprising:
a housing;
a power supply disposed within the housing; and
at least one electrode probe in electrical communication with the power supply and configured to make electrical contact to the patterned biosensor layer.

19. The dongle of claim 18, further comprising a spring configured to, in a first position, position the at least one electrode probe to contact the patterned biosensor layer, and, in a second position, to retract the at least one electrode probe.

20. The dongle of claim 19, where the dongle comprises an interface for electrical communication with a computing device.

21. A method for fabricating a biosensor device, the method comprising:
fabricating a vertical stack by:
providing a patterned biosensor layer;
forming a hydrogel layer over and in contact with the patterned biosensor layer;
forming a permeable metallic electrode over the hydrogel layer; and
forming a sample collection layer proximate and in contact with the permeable metallic electrode.

22. The method of claim 21, further comprising immobilizing a biomolecular ligand over and in contact with the patterned biosensor layer.

23. The method of claim 22, further comprising immobilizing at least two different types of biomolecular ligands over and in contact with the patterned biosensor layer.

24. The method of claim 23, wherein immobilizing the at least two different types of biomolecular ligands comprises:
a) applying at least two different types of capping agents to the patterned biosensor layer,
b) selectively removing one of the capping agents,
c) immobilizing a biomolecular ligand to the patterned biosensor layer,
d) repeating steps b) and c) at least once with a different type of biomolecular ligand.

25. A method for detecting an analyte in a sample, the method comprising the steps of:
depositing the sample onto a top surface of a biosensor device:
inserting the biosensor device into a measuring device;
activating the measuring device to induce the biosensor device to generate a signal indicating at least one of a presence or a quantity of the analyte; and
transmitting data describing the signal from the measuring device to a computing device.

26. The method of claim 25, wherein the biosensor device comprises:
a patterned biosensor layer.
a hydrogel layer disposed above and in contact with the patterned biosensor layer,
a permeable metallic electrode disposed above the hydrogel layer, and
a sample collection layer disposed proximate and in contact with the permeable metallic electrode.

27. The method of claim 25, wherein the measuring device comprises a dongle.

28. The method of claim 25, wherein activating the measuring device comprises at least one of applying an electrical signal to the biosensor device or illuminating the biosensor device.

29. The method of claim 25, wherein the computing device comprises a smartphone.
